(19) United States
(12) Patent Application Publication
(10) Pub. No.: US 2024/0226387 A1
(43) Pub. Date: Jul. 11, 2024

(54) DRUG COATING FOR EXPANDABLE BALLOON CATHETER AND PREPARATION METHOD THEREFOR

(71) Applicant: SHANGHAI BIO-HEART BIOLOGICAL TECHNOLOGY CO. LTD, Shanghai (CN)

(72) Inventors: Philip Li WANG, Shanghai (CN); Tao CAl, Shanghai (CN); Junyi WANG, Shanghai (CN); Hanyang LIU, Shanghai (CN); Chenzhao ZHANG, Shanghai (CN)

(21) Appl. No.: 18/240,363

(22) Filed: Aug. 31, 2023

Related U.S. Application Data
(63) Continuation of application No. PCT/CN2023/071403, filed on Jan. 9, 2023.

(51) Int. Cl. A61L 29/16; A61L 29/08

(52) U.S. Cl. CPC A61L 29/16; A61L 29/085; A61L 2300/606; A61L 2420/02

(57) ABSTRACT

The present disclosure belongs to the field of medical preparations, and particularly relates to a drug coating for an expandable balloon catheter and a preparation method therefor. The present disclosure selects two amphiphilic auxiliary materials of phospholipid and PEG, an outer layer of the phospholipid prevents a coating from being washed away by blood in a delivery process of a balloon, is dissolved with a cell membrane to realize a residence of a drug at a target position, and improves biocompatibility, and the hydrophilic excipient PEG enables a drug coating surface and a balloon surface to form an easy-to-strip state, such that the drug coating surface is separated from the balloon surface, and a balance of adhesion and stripping is realized.

### CROSS-REFERENCE TO RELATED APPLICATIONS

The present application is a continuation of International Application No. PCT/CN2023/071403, with an international filing date of Jan. 9, 2023, which is based upon and claims priority to Chinese Patent Application No. 202211657951.7, filed on Dec. 22, 2022, the entire contents of all of which are incorporated herein by reference.

### FIELD OF TECHNOLOGY

The present disclosure relates to the field of pharmaceutical preparations, in particular to the field of composite materials of a coated catheter under IPC No. A61L29/12, more specifically, to a drug coating for an expandable balloon catheter and a preparation method therefor.

### BACKGROUND

An expandable balloon is a novel device for an interventional therapy and conveys a drug to a target position in a form of a surface coated with a drug coating. After the expandable balloon reaches a destination, the balloon is inflated and expanded, such that the drug coating is tightly attached to the intima of a blood vessel, and a local effective release of the drug is realized. Currently, the expandable balloon coated with a drug coating is considered as one of a representative therapeutic regimen for “intervention and non-implantation”, has become “a place of strategic importance” of the medical device industry, and thus has a great market potential.

At present, the commercialized drug balloon is prepared by taking Sequent please developed by Braun company as an example, wherein paclitaxel is used as an active drug, iopromide is used as a carrier, and after a preparation, the drug is sprayed on a balloon surface. There are also Admiral Xtreme drug balloon, Lutonix035 drug balloon, etc., wherein paclitaxel is mostly used as an active drug, and different carrier technologies are used for preparing a drug coating. However, a traditional drug balloon has a low utilization rate of a drug. In a conveying process, the drug coating falls off from the balloon surface due to washing by blood, and most of the drug is lost before conveyed to a target position. Besides, in an expansion stage, a part of the drug coating still remains on the balloon surface, does not attached to a blood vessel wall, and is not released. In addition, since an interventional therapy has certain damage to a human body, multiple treatments may not be performed at short intervals and an effect of one treatment is limited, such that the traditional drug balloon has a great limitation, and a higher requirement is provided for a controllable sustained release of the drug coating.

The prior art CN 110292701B discloses a drug eluting balloon catheter. A drug coating is formed by mixing a phospholipid-coated drug and a hydrophilic excipient I as an inner layer and spraying a hydrophobic excipient II on an outer layer, thereby improving a utilization rate of a drug and realizing a rapid release of the drug. However, after the drug particle only wrapped by the phospholipid contacts with a blood vessel wall, the phospholipid is easy to fuse with a cell membrane, such that a burst release occurs, and an effect of a long-term sustained release may not be achieved.

### SUMMARY

Aiming at the defect of the prior art, the present disclosure aims to provide a drug coating for an expandable balloon catheter, which has a high drug utilization rate, is simple to prepare, and may realize a controllable drug release.

In another aspect, the present disclosure further aims to provide a preparation method for the drug coating for an expandable balloon catheter, which has a mild condition and a simple and convenient process.

In order to achieve the above objectives, the present disclosure uses the following technical solutions:

A drug coating for an expandable balloon catheter, comprising the following preparation raw materials in parts by weight: 20-80 parts of a drug, 10-70 parts of phospholipid, 5-60 parts of an excipient, and 5-60 parts of a copolymerized excipient.

Preferably, the drug is one or more selected from rapamycin, a rapamycin derivative, paclitaxel, heparin, and hirudin.

Preferably, the phospholipid is soybean phospholipid or lecithin.

Preferably, the excipient is one or more of polyethylene glycol (PEG), hyaluronic acid, chitosan, iopromide, shellac, tannic acid, polylactide, and a polylactic acid-glycolic acid (PLGA) copolymer.

Further preferably, the PEG has a number-average molecular weight of 8,000-30,000.

The present disclosure selects amphiphilic auxiliary materials, an outer layer of the phospholipid plays a protective role in blood, prevents a coating from being washed away by the blood in a delivery process of a balloon, and has a similar composition with a cell membrane. When the balloon is expanded, the coating is adhered to a blood vessel wall, the mutual dissolution of the phospholipid with the cell membrane to realize a residence of a drug at a target position, and improves biocompatibility. At this time, the hydrophilic excipient PEG absorbs water from the blood vessel, such that a drug coating surface and a balloon surface form an easy-to-strip state, the drug coating surface is separated from the balloon surface, and a balance of adhesion and stripping is realized.

Preferably, the copolymerized excipient is selected from one or more of an mPEG-PLGA block copolymer, a PEG-PLGA block copolymer, and a PEG-hyaluronic acid copolymer.

Preferably, a number-average molecular weight of methoxy polyethylene glycol (mPEG) in the mPEG-PLGA block copolymer is 200-8,000, and a number-average molecular weight of PLGA is 5,000-60,000. Further preferably, the mPEG in the mPEG-PLGA block copolymer is 550-5,000.

The present disclosure specifically selects a block copolymer consisting of the mPEG with a number-average molecular weight of 200-8,000, preferably 550-5,000 and the PLGA with a number-average molecular weight of 5,000-60,000 as the copolymerized excipient, which may form a wrapping on a surface of a drug droplet, and a controllable sustained release of a drug is realized by degrading a target position. The inventor has also surprisingly found that when the molecular weight of the mPEG is smaller, a drug release rate is more gentle; when the molecular weight of the mPEG is increased, the drug release rate is also correspondingly faster; and when the molecular weight of the mPEG reaches 8,000, an obvious burst release occurs. Based on the creative discovery, the drug coating of the present disclosure may adjust the drug release rate by adjusting the molecular weight of the mPEG in the copolymerized excipient, the mPEG-PLGA block copolymer, according to the actual use requirement and the drug property, thereby improving the universality of the drug coating.

Preferably, a molar ratio of the mPEG to the PLGA in the mPEG-PLGA block copolymer is (0.1-5):1, further preferably, 1:1.

Preferably, a mass ratio of the phospholipid to the excipient is (1-5):1.

Another aspect of the present disclosure provides a preparation method for the drug coating for an expandable balloon catheter, comprising the following steps:
weighing the drug, the phospholipid, the excipient, and the copolymerized excipient according to the parts by weight, dissolving same in an organic solvent, uniformly mixing same, and ultrasonically treating the mixture to obtain a drug micelle microsphere solution; and ultrasonically spraying the drug micelle microsphere solution onto a balloon surface of the expandable balloon catheter to obtain the drug coating.

Preferably, the organic solvent comprises one or more of ethanol, isopropanol, acetone, dichloromethane, trichloromethane, ethyl acetate, acetonitrile, butyl acetate, diethyl ether, and carbon tetrachloride.

Preferably, in the preparation method, the organic solvent is 15,000-25,000 parts by weight.

Preferably, the drug coating has a thickness of 1-10 μm.

Preferably, the drug micelle microsphere has a particle size less than or equal to 10 μm.

Compared with the prior art, the present disclosure has the following beneficial effects:

1. In the drug coating prepared by the present disclosure, an outer layer of the phospholipid prevents the coating from being washed away by blood in a delivery process of a balloon, is dissolved with a cell membrane to realize a residence of a drug at a target position, and improves biocompatibility.

2. The present disclosure selects the hydrophilic excipient PEG which absorbs water from the blood vessel, such that a drug coating surface and a balloon surface form an easy-to-strip state, the drug coating surface is separated from the balloon surface, and a utilization rate of the drug is further realized.

3. The drug coating of the present disclosure further comprises the mPEG-PLGA block copolymer with a specific molecular weight, creatively discovers an effect of the mPEG molecular weight on the drug release rate, and thus realizes a controllable release and a long-term slow release of a drug.

### BRIEF DESCRIPTION OF THE DRAWINGS

FIG. 1 is a scanning electron microscopy (SEM) image of the drug coating in example 1;

FIG. 2 shows drug release rates over time for the drug coatings prepared from the mPEG-PLGA block copolymers with mPEG molecular weights of 550 (example 1), 5,000 (example 2), and 8,000 (example 3) respectively; and

FIG. 3 shows drug release rates over time for the drug coatings prepared from the mPEG-PLGA block copolymers with mPEG molecular weights of 10,000 (comparative example 1) and 20,000 (comparative example 2).

### DESCRIPTION OF THE EMBODIMENTS

### Example 1

The present example provided a drug coating for an expandable balloon catheter, comprising the following preparation raw materials in parts by weight: 40 parts of rapamycin, 60 parts of soybean phospholipid, 40 parts of an excipient, 60 parts of a copolymerized excipient, and 20,000 parts of an organic solvent.

The excipient was PEG with a number-average molecular weight of 10,000 and purchased from Guangzhou Xilong Fine Chemical Technology Co., Ltd.

The copolymerized excipient was an mPEG-PLGA block copolymer, wherein a number-average molecular weight of mPEG was 550, a number-average molecular weight of PLGA was 60,000, a molar ratio of the mPEG to the PLGA was 1:1, and the mPEG-PLGA block copolymer was purchased from EVONIK (Shanghai).

The organic solvent was acetone.

The present disclosure further provided a preparation method for the drug coating for an expandable balloon catheter: the rapamycin, the soybean phospholipid, the PEG, and the mPEG-PLGA block copolymer were weighed according to the parts by weight, the materials were dissolved in the acetone and uniformly mixed, and the mixture was ultrasonically treated at 54 kHz for 30 min to obtain a drug micelle microsphere solution; and the drug micelle microsphere solution was ultrasonically sprayed on a balloon surface of the expandable balloon catheter, and drying was performed at 40° C. for 60 min to obtain the drug coating with a thickness of 10 μm.

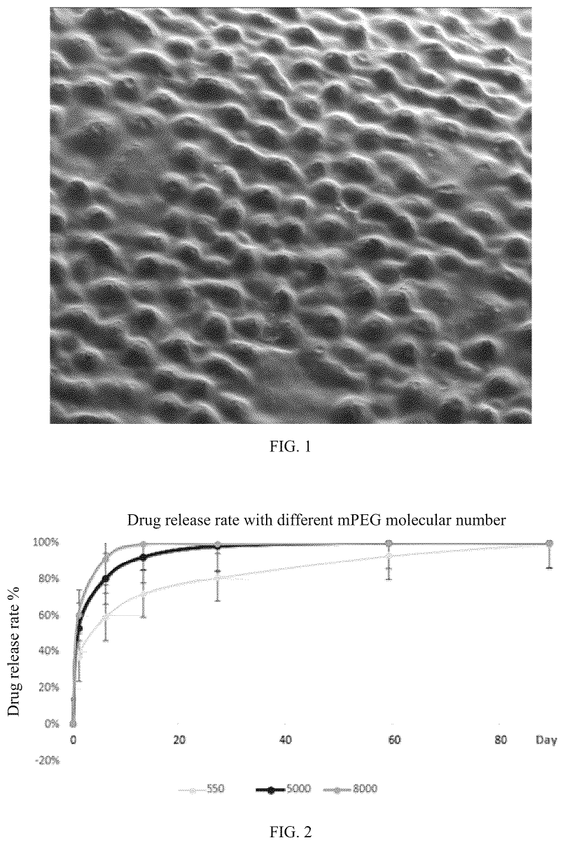

A drug micelle microsphere in the drug coating prepared in the example had an average particle size of 8 μm, the drug content of the coating was 7 μg/mm², and a scanning electron micrograph was shown in FIG. 1.

### Example 2

The present example provided a drug coating for an expandable balloon catheter and a preparation method therefor. The embodiment was the same as that in example 1, except that the copolymerized excipient was an mPEG-PLGA block copolymer, wherein a number-average molecular weight of mPEG was 5,000, a number-average molecular weight of PLGA was 60,000, a molar ratio of the mPEG to the PLGA was 1:1, and the mPEG-PLGA block copolymer was purchased from EVONIK (Shanghai).

A drug micelle microsphere in the drug coating prepared in the example had an average particle size of 10 μm, and the drug content of the coating was 6 μg/mm².

### Example 3

The present example provided a drug coating for an expandable balloon catheter and a preparation method therefor. The embodiment was the same as that in example 1, except that the copolymerized excipient was an mPEG-PLGA block copolymer, wherein a number-average molecular weight of mPEG was 8,000, a number-average molecular weight of PLGA was 60,000, a molar ratio of the mPEG to the PLGA was 1:1, and the mPEG-PLGA block copolymer was purchased from EVONIK (Shanghai).

A drug micelle microsphere in the drug coating prepared in the example had an average particle size of 5 μm, and the drug content of the coating was 8 μg/mm².

### Comparative Example 1

The present example provided a drug coating for an expandable balloon catheter and a preparation method therefor. The embodiment was the same as that in example 1, except that the copolymerized excipient was an mPEG-PLGA block copolymer, wherein a number-average molecular weight of mPEG was 10,000, a number-average molecular weight of PLGA was 60,000, a molar ratio of the mPEG to the PLGA was 1:1, and the mPEG-PLGA block copolymer was purchased from Jinan Jufukai Biotechnology Co., Ltd.

A drug micelle microsphere in the drug coating prepared in the example had an average particle size of 4 μm, and the drug content of the coating was 8 μg/mm².

### Comparative Example 2

The present example provided a drug coating for an expandable balloon catheter and a preparation method therefor. The embodiment was the same as that in example 1, except that the copolymerized excipient was an mPEG-PLGA block copolymer, wherein a number-average molecular weight of mPEG was 20.000, a number-average molecular weight of PLGA was 60,000, a molar ratio of the mPEG to the PLGA was 1:1, and the mPEG-PLGA block copolymer was purchased from Jinan Jufukai Biotechnology Co., Ltd.

A drug micelle microsphere in the drug coating prepared in the example had an average particle size of 4 μm, and the drug content of the coating was 8 μg/mm².

### Performance Test

Drug release testing: in order to verify a sustained-release effect of the drug coating prepared from the mPEG-PLGA block copolymers with different molecular weights, an animal experiment was used for testing a drug release rate of the drug coating obtained in each example. In this experiment, an animal model of mini-pigs was used, the drug coatings prepared in examples 1-3 and comparative examples 1-2 were used in mini-pigs respectively, and time nodes were set at 0 (instant) day, 1 day, 7 days, 14 days, 30 days, 60 days, and 90 days. A pig blood vessel was taken and subjected to a drug detection.

Experimental subject: the model of mini-pigs, weighed about 25-45 kg.

Experimental product: the drug coatings prepared from the mPEG-PLGA with different molecular weights in examples 1-3 and comparative examples 1-2 with a drug content of 1-10 μg/mm²

### Experimental Solution:

(1) 7 pigs were selected to each group of the animal model, corresponding to the time nodes of 0 (instant) day, 1 day, 7 days, 14 days, 30 days, 60 days, and 90 days respectively. Cardiac vessel RCA, LCX, and LAD of each group of the pig animal model were respectively implanted into a balloon of an expandable balloon catheter sprayed with the drug coatings in examples 1-3 and comparative examples 1-2.

(2) The pigs were sacrificed at the time nodes of 0 (instant) day, 1 day, 7 days, 14 days, 30 days, 60 days, and 90 days, and the blood vessel was taken for detecting the drug content.

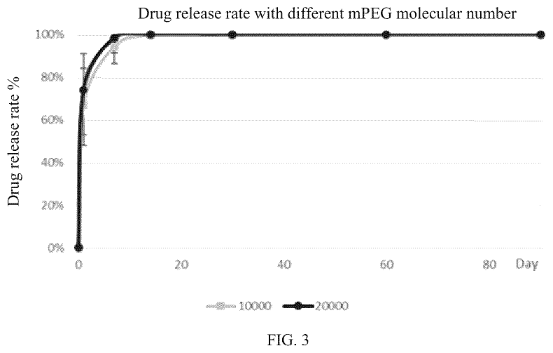

Experimental result: drug release curves were plotted according to the measured drug content (shown in FIGS. 2 and 3), and the drug release ratio on a y coordinate was a ratio of the content of the drug on a blood vessel wall to the content of the drug on the balloon surface before the implantation.

What is claimed is:

1. A drug coating for an expandable balloon catheter, comprising the following preparation raw materials in parts by weight: 20-80 parts of a drug, 10-70 parts of phospholipid, 5-60 parts of an excipient, and 5-60 parts of a copolymerized excipient.

2. The drug coating for an expandable balloon catheter according to claim 1, wherein the drug is one or more selected from rapamycin, a rapamycin derivative, paclitaxel, heparin, and hirudin.

3. The drug coating for an expandable balloon catheter according to claim 1, wherein the phospholipid is soybean phospholipid or lecithin.

4. The drug coating for an expandable balloon catheter according to claim 1, wherein the excipient is one or more of PEG, hyaluronic acid, chitosan, iopromide, shellac, tannic acid, polylactide, and PLGA.

5. The drug coating for an expandable balloon catheter according to claim 4, wherein a number-average molecular weight of the PEG is 8,000-30,000.

6. The drug coating for an expandable balloon catheter according to claim 1, wherein the copolymerized excipient is selected from one or more of an mPEG-PLGA block copolymer, a PEG-PLGA block copolymer, and a PEG-hyaluronic acid copolymer.

7. The drug coating for an expandable balloon catheter according to claim 6, a number-average molecular weight of mPEG in the mPEG-PLGA block copolymer is 200-8,000, and a number-average molecular weight of PLGA is 5,000-60,000.

8. The drug coating for an expandable balloon catheter according to claim 1, a mass ratio of the phospholipid to the excipient is (1-5):1.

9. A preparation method for the drug coating for an expandable balloon catheter according to claim 1, comprising the following steps:
weighing the drug, the phospholipid, the excipient, and the copolymerized excipient according to the parts by weight, dissolving same in an organic solvent, uniformly mixing same, and ultrasonically treating the mixture to obtain a drug micelle microsphere solution; and ultrasonically spraying the drug micelle microsphere solution onto a balloon surface of the expandable balloon catheter to obtain the drug coating.

10. The preparation method for the drug coating for an expandable balloon catheter according to claim 9, wherein the drug coating has a thickness of 1-10 μm.
